(19) United States
(12) Patent Application Publication
(10) Pub. No.: US 2025/0090449 A1
(43) Pub. Date: Mar. 20, 2025

(54) Foamed Microtablets for Oral Delivery of Active Ingredients

(71) Applicant: TSI Group Co., Ltd., Kiangsu Province (CN)

(72) Inventors: Joe Zhou, Missoula, MT (US); Spencer Zhang, Shanghai (CN); Lingwei Dai, Shanghai (CN)

(73) Assignee: TSI Group Co., Ltd., Kiangsu Province (CN)

(21) Appl. No.: 18/890,126

(22) Filed: Sep. 19, 2024

Related U.S. Application Data
(60) Provisional application No. 63/583,762, filed on Sep. 19, 2023.

(51) Int. Cl. A61K 9/46; A61K 9/00; A61K 9/20; A61K 31/191; A61K 31/7008; A61K 31/7076

(52) U.S. Cl. CPC A61K 9/0007; A61K 9/0056; A61K 9/2009; A61K 9/2018; A61K 31/191; A61K 31/7008; A61K 31/7076

(57) ABSTRACT

The present disclosure relates to orally ingested foamed dosage delivery forms. The present invention employs foamed microtablets to optimize the delivery of nutritional supplements, dietary supplements, functional foods, vitamins, minerals, amino acids, probiotics, herbal extracts and/or pharmaceuticals.

This application claims the benefit of U.S. Provisional Patent Application No. 63/583,762 filed Sep. 19, 2024 and herein incorporates U.S. Provisional Patent Application 63/583,762 by reference.

### BACKGROUND OF THE INVENTION

### 1. Field

The present disclosure relates to orally ingested foamed dosage delivery forms. The present invention employs foamed microtablets to optimize the delivery of nutritional supplements, dietary supplements, functional foods, vitamins, minerals, amino acids, probiotics, herbal extracts and/or pharmaceuticals.

### 2. Background

Traditional solid dosage delivery forms for nutritional supplements, vitamins, and pharmaceuticals can present difficulties to many people, including an inability or unwillingness to chew or swallow pills or tablets. If people have difficulty swallowing larger pills or tablets, smaller dosing capacities have to be employed to limit the size of the solid dosage form, which results in the need to consume the dosage in many pills or tablets. Slow dissolution times in the body also can result in poor absorption of the active ingredients. To overcome the issues with pills and tablets, new categories of delivery systems are increasing in consumer popularity, including powders, gummies, liquids and effervescence. Non-pill delivery forms still have significant shortcomings, including lower active ingredient levels and/or limited active loading capacity, weak shelf life and lack of convenience.

Effervescence is emerging as a non-pill delivery form that is popular with consumers. In a typical use of effervescent technology, a large effervescent tablet or a measured amount of effervescent powder containing active ingredients is dissolved in water or other liquids. Effervescent tablets usually contain acidic substances and carbonates or bicarbonates which react rapidly to release carbon dioxide when dissolved in liquid. One of the earliest commercial examples of effervescence for the delivery of active ingredients is Alka-Seltzer for the treatment of upset stomach. This traditional effervescent delivery form is a large tablet that is deposited into a liquid, such as water. The tablet dissolves, creating a bubbly beverage. The consumer drinks the beverage after the tablet has dissolved.

Effervescent formulations provide many benefits, including the ability to be quickly and uniformly absorbed when consumed. Traditional effervescent delivery forms, however, present problems described above, including lower active ingredient levels and/or limited active loading capacity, weak shelf life. In addition, some ingredients cannot be combined together in the same tablet. Further, traditional effervescent tablets have a high sodium content. Formulating effervescent compositions can be extremely difficult. The effervescent delivery form needs to dissolve relatively quickly and provide a product that is palatable to the consumer.

The microtablet products of the present invention can control the dissolving state of the product (dissolving at the bottom, surface or rolling for most of the time) by means of granulation of raw materials and auxiliary materials, partial granulation, and adjustment of water temperature during dissolution, and adjust the dissolution time of the product. Bubble retention time in the water, bottom or roll dissolution, and lower water temperature all significantly increase bubble retention.

The present invention provides foamed microtablets that overcome problems in the prior art. The microtablets of the present invention have a higher active loading quantity and/or multiple actives loading when compared to the prior art. In addition, active ingredients that are incompatible with each other can be separated into different single microtablets and packaged together. The microtablets of the present invention have a greater shelf life than traditional effervescent tablets. The microtablets of the present invention are easy to swallow and have improved taste over traditional effervescent tablets. Further, the present invention has a lower sodium content than prior art tablets. The microtablets of the present invention have less excipient quantity than in standard effervescent tablets.

The microtablets of the present invention provide a delivery form that is convenient, easy to administer, easy to swallow and tastes good.

Other objects, advantages and features of the present disclosure will become apparent from the following specification taken in conjunction with the accompanying figures.

### SUMMARY

The present invention is directed towards an effervescent composition in microtablet form. The microtablets can be granules, melts, or mints. The delivery form can be administered by dissolving the microtablets in liquid and then consuming the liquid or by placing the microtablet or microtablets directly in the mouth.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### DETAILED DESCRIPTION

The present disclosure relates to a novel delivery form for nutritional supplements, dietary supplements, functional foods, amino acids, probiotics, herbal extracts, vitamins, minerals and/or pharmaceuticals. The invention encompasses foamed microtablets, also referred to as microbeads, which can be administered to a person by various means, including direct dissolution in liquids and direct swallowing. The microtablet delivery form allows for formulation flexibility and higher amounts of active ingredients loaded into one serving. The foamed microtablets of the present invention offer the advantage of improved absorption of active agents. The microtablets are dissolved prior to ingestion instead of in the stomach. When delivery forms such as pills are ingested, slow dissolution times can lead to poor absorption of active ingredients.

Compositions of the present invention include active ingredients that are uniformly and accurately dispensed when dissolved in an aqueous medium, including water and/or other liquids, and/or saliva. The active ingredients are formed into effervescent microtablets, including granules, melts and/or mints which will act to enhance the delivery of active ingredients or combination of active ingredients.

### Definitions

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “actives” or “percent actives” or “percent by weight actives” or “actives concentration” are used interchangeably herein and refers to the concentration of those ingredients involved expressed as a percentage minus inert ingredients such as water or salts. It is also sometimes indicated by a percentage in parentheses, for example, “chemical (10%).”

The terms “active agent” or “active ingredient” refers to a component known to provide a health benefit when administered to an individual. Useful active agents include vitamins, amino acids, functional foods, pharmaceutical agents, minerals, probiotics (i.e., live microbials), dietary supplements, herbal extracts and combinations thereof. Suitable dietary supplements include (3-hydroxy-P-methylbutyrate (HMB), adenosine triphosphate (ATP), and glucosamine. “Dietary ingredient” includes vitamins and minerals herbs and other botanicals, amino acids, protein, dietary substances that are part of the food supply, such as enzymes and live microbials (commonly referred to as probiotics); and concentrates, metabolites, constituents, extracts or combinations of any dietary ingredient from the preceding categories.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-%, and in yet another embodiment, the amount of component is less than 0.01 wt-%.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used herein, “percent,” “%,” and the like are intended to be synonymous with “weight percent,” “wt-%,” etc.

The term “effervescent” or “effervescence” refers to the generation of bubbles in an aqueous media, including a reaction which generates bubbles, usually carbon dioxide, in aqueous media. Effervescence is due to an acid-base reaction in which the acid and base components need not be active agents in the oral dosage forms but must be present in sufficient quantities to produce effervescence. Effervescent oral dosage forms may contain other non-active ingredients and may contain binding agents.

The embodiments of this disclosure are not limited to particular methods and compositions which can vary and are understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components, or ingredients, but only if the additional steps, components, or ingredients do not materially alter the basic and novel characteristics of the claimed methods and compositions.

### Methods and Compositions

The microtablets of the present invention can have several forms, including granules, melts and mints. Granules or beads are typically 1 to 3 millimeters in size. Melts and mints, typically placed on the tongue or in the mouth, are somewhat larger.

The invention enables adjustable dissolution state and speed enabling precise control over dissolution characteristics based on raw material properties, including dissolution speed, foam volume and location of dissolution. Disintegration speeds are customized by adjusting the quantity of acid and alkali materials and altering the granulation process.

The invention allows for adjustable dissolution states and speeds, enabling precise control over dissolution characteristics such as speed, foam volume, and location. This is achieved through the management of raw material properties, including the bulk density of the material pile. For substances that dissolve slowly, such as amino acids, granulation can reduce the pile's density, allowing the product to remain in a rolling state longer, thus increasing its dissolution rate. Conversely, for plant extracts prone to foaming, avoiding granulation maximizes pile density, keeping the product at the bottom for a longer time to reduce foam.

For example, in a 2.5 g collagen formulation using the microtablets of the present invention, the foam height can reach 3 cm in a 250 mL cup, with a dissolution time of over 4.5 minutes. After granulating the collagen, the foam height is reduced to less than 1.5 cm, and the dissolution time shortens to under 3 minutes.

Dissolution speeds can also be tailored by adjusting the ratio of acid to alkali materials and modifying the granulation process. For instance, in a vitamin or mineral formulation of the present invention, with an acid-base ratio of approximately 32% (19% acid, 13% alkali), the disintegration time is typically 2 to 3 minutes. When the ratio is increased to about 47% (28% acid, 19% alkali), the disintegration time shortens to 50 seconds to 1.5 minutes. Adjusting granulation alone, without changing the acid-base ratio, can reduce disintegration time by 10-20 seconds.

For the present invention, key adjustment methods include several options, including adding excipients during granulation to improve the dissolution characteristics of raw materials. For example, slow-dissolving substances like extracts, amino acids, or collagen may require 20-50% excipients (e.g., sugar alcohols) to promote dissolution, while common ingredients like minerals or vitamins typically need 4-20% excipients for better fluidity and solubility.

Another adjustment includes modifying the granulation process for raw materials, effervescent agents, or excipients to adjust the bulk density and optimize the product's dissolution behavior (e.g., rolling or sinking). For example, a multi-vitamin tablet without granulation may have a bulk density of 0.95-1.05 g/mL, leading to disintegration at the bottom for 1 minute, rolling dissolution for 30 seconds, and floating dissolution within 2 minutes. When minerals and acids are granulated, the bulk density drops to 0.7-0.8 g/mL, shortening the total disintegration time to about 1 minute 20 seconds.

In addition, a further modification includes adjusting the ratio and type of effervescent agents to fine-tune the dissolution rate. Increasing the proportion of effervescent agents (citric acid-sodium bicarbonate ratio in the microtablet products of the present invention typically ranges from 35-40%) enhances the reaction and accelerates disintegration. For example, in a multi-vitamin microtablet formulation with 35% effervescent agents, the dissolution time is about 2 minutes 20 seconds. Lowering the effervescent agent content to 28% extends the dissolution time to 3 minutes, while increasing it to 55% shortens it to 1 minute 10 seconds. Additionally, substituting malic acid for citric acid extends dissolution time by 5-15%, as malic acid reacts slower. Sodium carbonate and potassium bicarbonate can replace sodium bicarbonate, extending dissolution by 10-20% and 5-10%, respectively, but their higher density helps control rolling behavior.

The method of the present invention can precisely control the dissolution behavior of the microbeads (e.g., dissolving at the bottom, on the surface, or predominantly rolling) by adjusting the granulation of raw and auxiliary materials. Granulation techniques, including partial granulation, help regulate the bulk density of the product pile. For example, complete granulation reduces the bulk density, allowing the micro-tabs to float more easily. When the granulation process is adjusted, the rate of dissolution can be modified.

By way of example, in the case of a multi-vitamin and mineral formulation with no granulation, the bulk density is between 0.95-1.05 g/mL. Initially, the product stays at the bottom of the cup for about 1 minute, then gradually rolls and dissolves for 30 seconds before floating and dissolving completely.

With mineral granulation, the bulk density decreases to 0.8-0.9 g/mL, resulting in bottom dissolution for 45-55 seconds, rolling dissolution for 40 seconds, and then floating to the top.

When both minerals and acids are granulated, the bulk density is further reduced to 0.7-0.8 g/mL. In this case, bottom dissolution lasts 20-30 seconds, followed by rolling dissolution for 30-50 seconds before floating.

Additionally, adjusting water temperature impacts the dissolution rate. For example, at 15-20° C., the dissolution time is approximately 1 minute and 30 seconds. At 25-30° C., it shortens to around 50 seconds, while at 5-10° C., it extends to about 2 minutes. Bubble retention in water, bottom or rolling dissolution, and lower temperatures significantly increase the duration of bubble retention.

These adjustment methods include but are not limited to: 1) Participate in granulation with a certain amount of excipients to improve the characteristics of raw materials; 2) Granulation or partial granulation of raw materials, effervescent agents or excipients to adjust the density of finished products during dissolution to meet the needs 3) Adjustment of effervescent agent ratio, effervescent agent diversity selection and effervescent agent post-processing methods, etc.

The tablets may be formulated such that there is a range of dissolution times, including dissolution in a liquid in around 5 seconds to around 5 minutes. The tablets may be dissolved in a drinkable liquid with fizzy effects. The level of fizziness can be controlled by the amount of carbon dioxide or hydrogen generated during the dissolution process.

Tablets may also be formed to be dissolved completely in the mouth in a time of around 5 seconds to around 10 minutes. These tablets can create a fizzy effect in the mouth by the release of carbon dioxide or hydrogen. The amount of carbon dioxide or hydrogen can be controlled by the adjustment of the formula. As shown in the reaction equation, 3 molecules of sodium bicarbonate (NaHCO₃) react with 1 molecule of citric acid (C₆H₈O₇) to produce 3 molecules of carbon dioxide (CO₂), 3 molecules of water (H₂O), and sodium citrate (C₆HSO₇Na₃): 3NaHCO₃+C6H8O7→C6H5O7Na3+3H2O+3CO2↑ Similarly, 2 molecules of sodium (Na) react with water to produce 1 molecule of hydrogen gas (H₂) and sodium hydroxide (NaOH):2Na+2H2O→2NaOH+H2↑

Using these equations, it is possible to calculate the required amounts of sodium bicarbonate or sodium based on the target amount of CO₂ or H₂ gas. Typically, 0.012 mol of CO₂ or H₂ is needed in 200 mL of water to achieve a bubble level similar to that of soda. However, about 80% of the bubbles escape, leaving 20% retained to reach the desired effect. Based on this, we can calculate that the required amount of sodium bicarbonate is:

$$0.012 mol \times 84.01 g / mol = 1 g$$

For sodium, the required amount is:

$$0.012 mol \times 2 \times 23 g / mol = 0.55 g$$

The microtablets of the present invention contain an acid and an alkali. Acids include citric acid, ascorbic acid, malic acid, tartaric acid, adipic acid, fumaric acid, lactic acid, acetic acid, oxalic acid.

Alkalis include the following:

### Carbonates:

Sodium carbonate (Na₂CO₃)
Calcium carbonate (CaCO)
Potassium carbonate (K₂CO₃)
Magnesium carbonate (MgCO₃)
Lithium carbonate (Li₂CO₃)
Iron(I) carbonate (FeCO₃)
Copper(II) carbonate (CuCO₃)
Zinc carbonate (ZnCO₃)

### Bicarbonates:

Sodium bicarbonate (NaHCO₃)
Potassium bicarbonate (KHCO₃).
Calcium bicarbonate (Ca(HCO₃)₂)
Magnesium bicarbonate (Mg(HCO₃)₂)
Ammonium bicarbonate (NH₄HCO₃)

A new oral delivery form is provided that can be administered by (1) dissolving in a liquid and then consumed orally, or (2) directly placed in the mouth and dissolved in the mouth. This dosage form provides an individual with convenient and flexible choices to administer dietary supplements, functional foods and/or pharmaceutical preparations.

The dosage form can be provided in many sizes, shapes and weights, including tablets with diameters ranging from 0.5 mm to 20 mm and weights from 0.00 to 20 grams.

The microtablets of the present invention have a higher serving size, so active ingredients can be loaded in higher amounts Traditional effervescent tablets are limited by tablet weight, so active loading quantities have an upper limit. The microtablets of the present invention have a significantly higher active loading quantity. Active ingredient loading limits can be greater than 40% by weight. Active ingredients can be incorporated into a single serving of microtablets from 50 mg up to 100 grams. In effervescent tablets, the typical amount of effervescent agents (acid and alkali) ranges from 55-85%, whereas in formulations of the present invention, it is usually between 35-45%. This allows formulations of the present invention to have more available space to incorporate active ingredients, resulting in a higher payload capacity.

In terms of dosage, commercially available effervescent tablets generally weigh between 3.5-5.5 g per tablet, which limits the amount of active ingredients that can be included. In contrast, formulations of the present invention are more flexible and can be scaled up to larger serving sizes, such as 10 g, 20 g, or even 30 g, allowing for a greater quantity of active ingredients to be delivered.

By way of example, the active ingredient HMB is limited to 500 mg in a traditional effervescent tablet. When HMB is used as the active ingredient in the present invention, the microtablets can be administered in any quantity, so the dosing level is not limited to 500 mg. A serving can include 1000 mg, 2000 mg, 3000 mg or more of HMB. In the traditional delivery form for HMB which is pills, an individual may desire to take 3 grams per day, requiring the consumption of three very large pills each containing 1 gram of HMB. Using the delivery system of the present invention, an individual may consume the entire 3 grams of HMB in one serving of microtablets. The one serving can be dissolved in a liquid and the liquid then consumed, or the microtablet(s) may be placed directly into the mouth and dissolved there.

The higher active loading capability of the present invention results in the ability to reduce the number of servings required per day in order to achieve an efficacious dose. This is significantly more convenient for the consumer and much easier to use to achieve the efficacious dose. In effervescent tablets, the amount of effervescent agents (acid and alkali) typically ranges from 55-85%, while in the formulations of the present invention, it is generally between 35-45%. This difference allows formulations of the present invention to have more space for loading active ingredients, thus increasing their payload capacity.

Additionally, commercial effervescent tablets are usually limited to a weight of 3.5-5.5 g per tablet, which restricts the amount of active ingredients that can be included. In contrast, formulations of the present invention are not limited by the weight of each serving and can be scaled up to 10 g, 20 g, or even 30 g+, allowing for significantly higher doses of active ingredients.

This makes the microtablets of the present invention particularly suitable for formulations requiring higher daily doses, such as collagen (3-10 g/day) or oral rehydration salts (5-20 g/day of glucose, sodium, and potassium). For instance, the microtablets of the present invention can easily accommodate larger formulations, such as rehydration products exceeding 30 g per serving.

Each microtablet may contain one or more active ingredients. Active ingredients include but are not limited to dietary supplements, functional foods, nutraceuticals, vitamins, minerals, enzymes, probiotics, herbal extracts, and/or pharmaceuticals. Microtablets with different actives can be packed together, resulting in customization of ingredients and quantity. Functional or active ingredients are paired with distinct flavors, colors, sweeteners, resulting in administration of precisely controlled and customizable actives and customized sensory diversity. The customized disintegration speed means that the microtablets are easy to consume and are quickly absorbed. The microtablets provide an innovative sensation experience.

The microtablets of the present invention have a greater shelf life than that of traditional effervescent tablets. The longer shelf life is due to the lower weight percentage of acid and alkali materials, resulting in microtablets that are less sensitive to moisture and heat than prior effervescent tablets. In traditional effervescent tablets, the total amount of acid (28-40%) and alkali (25-38%) typically exceeds 60% of the tablet's weight. Due to the high content of these components, effervescent tablets generally require packaging in tubes with desiccants in the cap to maintain stability throughout their shelf life.

In contrast, formulations of the present invention contain lower quantities of acid (16-25%) and alkali (13-19%), with the total amount of effervescent agents making up around 40% of the product's weight. This reduced acid and alkali content enhances the safety of formulations of the present invention and allows for packaging in aluminum foil bags without the need for desiccants. Additionally, the microtablets of the present invention exhibit better stability compared to traditional effervescent tablets during accelerated testing (40° C., 75% RH), which is attributed to the lower effervescent agent content.

The microtablets of the present invention can be stored at room temperature. The invention permits the adjustment of bubble volume during administration, achieved through formulation adjustments, process optimization and effervescent agent manipulation.

Single-component particles can be created, which can be combined with different flavors, colors and sweeteners to create precision nutrition and enhance consumption efficiency. Functional ingredients delivered in the microtablet format include but are not limited to anti-aging, anti-inflammatory, detox, energy booster, eye health, gut health, immunity, joint, minerals, vitamins, herb extracts, muscle health, skin, hair, nails, sleep, stress, and/or urinary health ingredients. The ability to create single-component particles results in the ability to personalize the formulation. By way of example, a personalized formulation can be created by including particles containing customized combinations of ingredients in amounts and ingredients targeted to a particular individual's needs.

Some active ingredients cannot be combined together within the same delivery form, including within the same effervescent tablet. The present invention overcomes this issue by allowing incompatible ingredients to be loaded onto separate particles and then packed together. An individual can consume all of the active ingredients in one serving. By way of example, vitamin C (Vc) reduces Fe³⁺ to Fe²⁺, which introduces significant uncertainties during product production and storage. This can lead to issues such as product discoloration (due to Vc oxidation or changes in iron's oxidation state), even if Fe²⁺ is initially added. This reaction can also interfere with the accurate detection of iron content, complicating quality control measures and compromising the product's stability and appearance.

The present invention allows for the combination of raw materials with different properties or poor stability at low concentrations. For example, proteins with specific functions (such as enzymes) are stable at the mg/ml level and will be significantly degraded at the pg/ml level. The microtablets of the present invention can be used to achieve high concentrations in a single tablet. The tablets of the present invention also work well for substances with low detection sensitivity or low recovery.

A serving dosage can be a single tablet or multiple tablets. The range of tablets in a dose may be 1 tablet to 2000 tablets. The tablets in a single dose can be packaged for convenient serving of a single dose, including as a sachet, tube, bottle, pod, straw, etc. The tablets may contain one formulation of tablet or different types of tablets (for example, tablets with different active ingredients) packed together.

The serving package with one or more types of tablets are precisely packed via precision dispensing technologies.

The microtablets of the present invention allow for higher active ingredient loading while reducing the need for excessive excipients which enhances consumption efficacy. For conventional materials, formulations using the microtablets of the present invention can achieve a drug load of 50-60%. However, for materials prone to foam production, such as plant extracts or collagen, the drug load may be adjusted to 30-45%. For rehydration salt products, the drug load can reach up to 80%, since sodium bicarbonate also serves as an active component.

In traditional effervescent tablets, the effervescent agents (acid and alkali) typically comprise 55-85% of the formulation. In contrast, the microtablet formulations of the present invention use only 35-45% effervescent agents, allowing for a larger proportion of the formulation to be dedicated to active ingredients. This results in a higher drug load and more effective space utilization, making the microtablets of the present invention advantageous for incorporating higher levels of active substances.

One issue with prior art effervescent technology is the high sodium content required by large effervescent tablets. In the present invention, the sodium content can be lowered while also ensuring consistent disintegration time due to precise coordination between acid and alkali additives. The microtablets of the present invention doses currently exceed 4.5 g per serving and can be adjusted based on the amount of active ingredients, with possible doses of 5 g, 10 g, or 15 g. Compared to traditional effervescent tablets, The microtablets of the present invention includes films are smaller and allow for adjustments in the rolling state, which results in a relatively faster dissolution rate. This means that the microtablets of the present invention require less effervescent agent: for a 5 g dose, The microtablets of the present invention typically contain 13-20% effervescent agents, corresponding to a sodium content of 177-273 mg. In contrast, conventional effervescent tablets contain 25-38% effervescent agents, resulting in a sodium content of 342-520 mg.

Packaging of effervescent tablets can provide challenges. The microtablets of the present invention can be packaged in bar packs, aluminum cups, combination packaging and individual packaging of multiple components. The microtablets can be contained in easy-to-carry cups, such as pods, and can also be packaged in straws, and/or sachets. Packaging the microtablets in this way provides premeasured doses, eliminating the need for the consumer to measure out each serving. This ensures that the consumer receives an exact dosage of the active ingredients.

The microtablets of the present invention can be administered through a variety of routes, including direct dissolution in water or other beverages and/or liquids, direct oral ingestion of the microtablets, combinations of dissolution in liquid and oral ingestion, or direct swallowing.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of the present disclosure, and without departing from the spirit and scope thereof, can make various changes and modifications of the embodiments to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

Effervescent microtablet technology makes it easier to pack more active ingredients into a small, fast-acting supplement, improving convenience, bioavailability, and overall effectiveness. The following nonlimiting examples describe a variety of formulations, including a post-workout formulation, an energy formulation and an immune support formulation.

### Example 1

A post-workout formulation is provided in microtablet form. This post-workout supplement is in effervescent microtablet form, providing a high concentration of active ingredients in a single-serving sachet. The effervescent action helps nutrients dissolve quickly, making it ideal for fast recovery after intense exercise.
Magnesium (as Magnesium oxide) 300 mg
HMB 1.5 g
Creatine monohydrate 3 g
Potassium (as Potassium sulfate) 200 mg
Calcium (as Calcium hydrogen phosphate) 250 mg
   In this example, there are several key benefits of the effervescent microtablet technology:
1. Higher Active Load: Microtablets allow for larger doses of ingredients like HMB and Creatine, which would be difficult to fit into regular effervescent tablets due to space limits from effervescent agents like sodium bicarbonate.
2. Faster Absorption: The effervescent action quickly dissolves the microtablets in water, promoting faster absorption of key nutrients, ideal for immediate post-workout recovery.
3. Convenient Sachets: Single-serving sachets offer easy, on-the-go usage, and make consumption simpler compared to bulky powders or hard-to-swallow tablets.
   Effervescent microtablets improve stability, shelf life, and user experience by providing a convenient, pleasant, and fast-acting supplement for athletes and fitness enthusiasts.

### Example 2

A bone, energy and immunity formulation is provided in microtablet form:

| | | | |
|---|---|---|---|
| Ascorbic Acid | 1000 | mg | Provides |
| | | | robust |
| | | | immune |
| | | | support, |
| | | | enhances |
| | | | collagen |
| | | | production for |
| | | | bone health, |
| | | | and functions |
| | | | as a powerful |
| | | | antioxidant |
| Thiamine Nitrate | 15 | mg | Essential for |
| | | | energy |
| | | | production |
| | | | and cellular |
| | | | function |
| Riboflavin | 1 | mg | Supports |
| | | | energy |
| | | | production |
| | | | and cellular |
| | | | function |
| Pyridoxine Hydrochloride | 2 | mg | Aids in amino |
| | | | acid |
| | | | metabolism |
| | | | and brain |
| | | | health |
| Nicotinamide | 50 | mg | Facilitates |
| | | | carbohydrate, |
| | | | fat, and |
| | | | protein |
| | | | metabolism. |
| Calcium Pantothenate | 23 | mg | Contributes to |
| | | | energy |
| | | | metabolism |
| | | | and synthesis |
| | | | of coenzyme |
| | | | A. |
| Folic Acid | 400 | mcg | Vital for |
| | | | DNA |
| | | | synthesis and |
| | | | red blood cell |
| | | | formation |
| Biotin | 150 | mcg | Supports skin, |
| | | | hair, and nail |
| | | | health, as well |
| | | | as energy |
| | | | metabolism |
| Cyanocobalamin (Vitamin | 10 | mcg | Necessary for |
| B12) | | | red blood cell |
| | | | production |
| | | | and |
| | | | neurological |
| | | | function. |
| Zinc Citrate Dihydrate | 10 | mg | Supports |
| | | | immune |
| | | | function and |
| | | | antioxidant |
| | | | defense |
| Magnesium (as Magnesium | 120 | mg | Essential for |
| Carbonate Hydrate) | | | bone health, |
| | | | muscle |
| | | | relaxation, |
| | | | and energy |
| | | | production |
| Calcium (as Calcium | 150 | mg | Crucial for |
| Carbonate) | | | maintaining |
| | | | strong bones |
| | | | and teeth, and |
| | | | for muscle |
| | | | contraction. |

### Key Benefits of Effervescent Microtablet Technology:

1. High Active Ingredient Load: Effervescent microtablets allow for the inclusion of large doses of essential nutrients, such as 1000 mg of Vitamin C and multiple B-vitamins, in a compact form. This is especially advantageous compared to traditional effervescent tablets, which have limitations due to the space taken up by effervescent agents.
2. Fast Dissolution & Absorption: The effervescent technology enables the microtablets to dissolve rapidly in water, ensuring that nutrients like Vitamin C and Magnesium are absorbed quickly and effectively, which is beneficial for immediate post-workout recovery and overall health support.
3. Convenient Sachets: Each microtablet serving is packaged in a single-serving sachet, making it easy to carry and use. This format is travel-friendly and eliminates the need for measuring or handling bulky powders, offering a practical solution for maintaining health on the go.

### Example 3

This immunity and mobility support supplement is delivered in effervescent microtablet form, allowing for the inclusion of a diverse range of active ingredients in a single-serving sachet. The effervescent technology enhances nutrient dissolution and absorption, making it an effective option for supporting immune health and joint mobility.

| | | | |
|---|---|---|---|
| Ascorbic Acid | 24 | mg | Supports |
| | | | immune |
| | | | function and |
| | | | acts as an |
| | | | antioxidant |
| Vitamin D3 | 200 | IU | Essential for |
| | | | bone health |
| | | | and immune |
| Zinc citrate dihydrate | 32.6 | mg | Aids in |
| | | | immune |
| | | | function and |
| | | | antioxidant |
| | | | defense |
| Astragalus membranaceus | 800 | mg | Known for its |
| dry Root | | | immune- |
| | | | boosting and |
| | | | antiviral |
| | | | effects |
| Citrus bioflavonoids | 10 | mg | Supports |
| | | | antioxidant |
| | | | activity and |
| | | | enhances the |
| | | | effectiveness |
| | | | of Vitamin C. |
| Pomegranate juice | 720 | mg | Provides |
| | | | antioxidants |
| | | | and supports |
| | | | joint health |
| Sambucus nigra fruit juice | 500 | mg | Known for its |
| | | | immune- |
| | | | boosting and |
| | | | antiviral |
| | | | effects |
| Rosa canina dry | 400 | mg | Rich in |
| | | | vitamin C and |
| | | | supports joint |
| | | | health |
| Zingiber officinale root dry | 500 | mg | Offers anti- |
| | | | inflammatory |
| | | | benefits and |
| | | | supports |
| | | | mobility |
| Boswellia serrata dry | 500 | mg | Supports joint |
| | | | health and |
| | | | reduces |
| | | | inflammation |
| Methyl Sulfonyl Methane | 50 | mg | Promotes |
| | | | joint health |
| | | | and reduces |
| | | | inflammation |

### Key Benefits of Effervescent Microtablet Technology:

1. Higher Active Ingredient Load: Effervescent microtablets accommodate a high concentration of diverse ingredients, such as Astragalus and Boswellia, that would be challenging to fit into conventional effervescent tablets due to space constraints.
2. Enhanced Absorption: The effervescent action facilitates the rapid dissolution of the microtablets, allowing for quicker absorption of nutrients like Vitamin C, Zinc, and herbal extracts, which is crucial for effective immune support and joint mobility.
3. Convenient Sachets: Packaged in single-serving sachets, the microtablets are easy to carry and use, offering a user-friendly solution compared to bulky powders or large tablets. This format ensures you get your nutrients quickly and conveniently, anywhere.
   In Example 3, where the total quantity of herbal extract exceeds 1 g, dissolving in a 250 ml cup can cause foam overflow (foam height >35 mm).
   Formulation Optimization: To address this, it's important to carefully evaluate the choice of effervescent agents. The typical combination of citric acid and sodium bicarbonate (84 g/mol) may need adjustment. By incorporating higher-density alkaline materials, such as sodium carbonate (106 g/mol) and potassium bicarbonate (100 g/mol), you can prolong the disintegration time at the bottom of the cup and reduce floating, which in turn minimizes foam generation at the top.
   Process Optimization: Granulating the herbal extract can also be beneficial. For example, adding 40% sugar alcohols (like sorbitol or mannitol), which dissolve well, will facilitate the dissolution of the extract when granulated together.
   Control of Effervescent Agents: If the effervescent agent content is too high (45-50%), the reaction will be too vigorous, leading to more foam. Reducing the effervescent agent content to a more moderate range of 35-40% will help control the foam production, extend the dissolving time, and lower the foam height.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A solid effervescent dosage form comprising:
a plurality of minitablets, wherein each of the plurality of minitablets comprises:
one or more active agents;
one or more acids;
one or more alkaline agents;
one or more binders and/or excipients; and
wherein each of the plurality of minitablets dissolves when placed in an aqueous solution producing an effervescent aqueous solution and releasing the one or more active agent into the effervescent aqueous solution.

2. The dosage form of claim 1, wherein one or more active agent is selected from dietary supplements, vitamins, minerals, herbs, botanicals, amino acids, proteins, enzymes, probiotics, and concentrates, metabolites, constituents, extracts, or combinations of any dietary ingredient, functional foods, pharmaceutical agents, or combinations thereof.

3. The dosage form of claim 2, wherein the one or more active is O-hydroxy-p-methylbutyrate (HMB), adenosine triphosphate (ATP), and/or glucosamine, or combinations thereof.

4. The dosage form of claim 1, wherein the one or more active agent confers a benefit selected from: anti-aging, anti-inflammatory, detox, energy booster, eye health, gut health, immunity, joint, minerals, vitamins, herb extracts, muscle health, skin, hair, nails, sleep, stress, and/or urinary health ingredients.

5. The dosage form of claim 1, wherein the plurality of minitablets further comprise a first plurality of minitablets and a second plurality of minitablets, wherein the one or more active agent in the first plurality of minitablets is different from the second plurality of minitablets.

6. The dosage form of claim 1, wherein the one or more active agents comprises at least about 40% (wt/wt) of the minitablets.

7. The dosage form of claim 1, wherein the one or more acids is selected from citric acid, tartaric acid, malic acid, fumaric acid, ascorbic acid, lactic acid, adipic acid, succinic acid, gluconic acid, acetic acid, phosphoric acid, and combinations thereof.

8. The dosage form of claim 1, wherein the one or more alkaline agents is selected from: Sodium carbonate, Calcium carbonate, Potassium carbonate, Magnesium carbonate, Lithium carbonate, Iron(I) carbonate, Copper(II) carbonate, Zinc carbonate, Sodium bicarbonate, Potassium bicarbonate, Calcium bicarbonate, Magnesium bicarbonate, Ammonium bicarbonate and combinations thereof.

9. The dosage form of claim 1, wherein the molar ratio of the acid to the alkaline agent is from about 0.1 to 10.

10. The dosage form of claim 1, wherein each of the plurality of minitablets has a diameter of from about 0.5 mm to about 20 mm.

11. The dosage form of claim 10, wherein each of the plurality of minitablets has a diameter of from about 1 mm to about 3 mm.

12. The dosage form of claim 1, wherein each of the plurality of minitablets further comprises a colorant.

13. The dosage form of claim 1, wherein each of the plurality of minitablets further comprises a flavorant.

14. The dosage form of claim 1, wherein each of the plurality of minitablets dissolves within an aqueous solution in from about 5 seconds to about 5 minutes.

15. The dosage form of claim 1, wherein the sodium wt % of each tablet does not exceed about fifty percent.

16. The dosage form of claim 1, wherein each of the plurality of minitablets further comprises a sugar alcohol.

17. The dosage form of claim 16, wherein the sugar alcohol is sorbitol or mannitol.

18. The dosage form of claim 1, wherein the dosage form is packaged in a sachet, tube, bottle, pod, stick, blister pack, jar, or straw.

19. The dosage form of claim 1, wherein dosage form is packaged as a single serving.

20. A solid effervescent dosage form comprising:
a minitablet comprising:
one or more active agents;
one or more acids;
one or more alkaline agents;
one or more binders and/or excipients; and
a flavorant and/or flavorants
and wherein the minitablet dissolves when placed in an aqueous solution producing an effervescent aqueous solution and releasing the one or more active agent into the effervescent aqueous solution.

21. The dosage form of claim 20, wherein the flavorant is mint.

22. The dosage form of claim 20, wherein the aqueous solution is saliva.

23. The dosage form of claim 20, wherein the weight per minitablet is between about 5 mg to 500 mg.

24. The dosage form of claim 23, wherein the weight per minitablet is between about 20 mg to 100 mg.
